(19) United States
(12) Patent Application Publication
(10) Pub. No.: US 2016/0184571 A1
(43) Pub. Date: Jun. 30, 2016

(54) Prefilled Syringe Devices Employing Microneedle Interfaces for Intradermal Delivery

(71) Applicant: NANOPASS TECHNOLOGIES LTD., Nes Ziona (IL)

(72) Inventor: Gal ADMATI, Kibbutz Dorot (IL)

(21) Appl. No.: 14/903,061

(22) PCT Filed: Sep. 9, 2014
(86) PCT No.: PCT/IL2014/050801

§ 371 Date: Jan. 6, 2016

Related U.S. Application Data
(60) Provisional application No. 61/875,131, filed on Sep. 9, 2013.

(51) Int. Cl. A61M 37/00; A61M 5/31; A61M 5/34; A61M 5/24

(52) U.S. Cl. CPC A61M 37/0015; A61M 5/2466; A61M 5/31; A61M 5/34; A61M 2037/0023

(57) ABSTRACT

A device (10) for delivering a liquid into the skin of a patient has a syringe (12) containing a liquid, and a septum (18) attached to the syringe in sealing relation to a fluid flow outlet (16). A microneedle adapter (20), interengaged with the syringe, has one or more hollow microneedles (22) for insertion into the skin of the patient, and a septum-penetrating hollow projection (24) in facing relation to the septum. When the microneedle adapter (20) is in a storage position, the fluid flow outlet (16) is sealed by the septum (18) for storage of the contained liquid. The microneedle adapter (20) is displaceable to an actuated position in which the septum-penetrating hollow projection (24) penetrates the septum (18), thereby opening a continuous fluid flow path for the liquid from the syringe through the microneedle adapter for injection through the hollow microneedles. An alternative device (100) employs a septum-penetrating projection (116) integrated with an injection molded syringe body (12).

### FIELD AND BACKGROUND OF THE INVENTION

The present invention relates to prefilled syringe devices employing microneedle interfaces for intradermal delivery.

Prefilled syringes (PFS) are used for long term storage and delivery of substances, in various sites such as the subcutaneous or intramuscular tissues. Typically, prefilled syringes are made of glass, which provides sufficient impermeability and stability for long term storage of drugs. Certain polymers have recently been approved for use in prefilled syringes, such as cyclic olefin polymer (COP) and cyclic olefin copolymer (COC).

There are particular advantages to preassembled prefilled syringe devices, where the needle is provided as part of the sterile device, ready for use, simplifying use and typically reducing dead-space wastage (i.e., the remaining liquid at the end of injection).

It has been proposed to provide a microneedle injection interface with a prefilled syringe, allowing the advantages of prefilled syringes together with the capability of true intradermal drug delivery. A proposed device is disclosed in PCT Patent Application Publication No. WO 2012/098503 A1.

### SUMMARY OF THE INVENTION

The present invention is a prefilled syringe device employing a microneedle interface for intradermal delivery.

According to the teachings of an embodiment of the present invention there is provided, a device for delivering a liquid into the skin of a patient, the device comprising: (a) a syringe containing a liquid, the syringe including a fluid flow outlet; (b) a septum attached to the syringe in sealing relation to the fluid flow outlet; and (c) a microneedle adapter interengaged with the syringe so as to be displaceable from a storage position to an actuated position, the microneedle adapter comprising: (i) at least one hollow microneedle deployed for insertion into the skin of the patient, and (ii) a septum-penetrating hollow projection deployed in facing relation to the septum, wherein the microneedle adapter defines a flow path from the septum-penetrating hollow projection to the at least one hollow microneedle, wherein, when the microneedle adapter is in the storage position, the fluid flow outlet is sealed by the septum and, when the microneedle adapter is displaced to the actuated position, the septum-penetrating hollow projection penetrates the septum, thereby opening a continuous fluid flow path for the liquid from the syringe through the microneedle adapter for injection through the at least one hollow microneedle.

According to a further feature of an embodiment of the present invention, the septum is attached to the syringe by a connecting collar, and wherein the microneedle adapter is interengaged with the syringe by engagement with the connecting collar.

According to a further feature of an embodiment of the present invention, the septum is integrated with the connecting collar by overmolding.

According to a further feature of an embodiment of the present invention, the microneedle adapter is interengaged with the syringe via a threaded engagement such that rotation of the microneedle adapter about a longitudinal axis of the syringe generates axial motion of the microneedle adapter from the storage position to the actuated position.

According to a further feature of an embodiment of the present invention, the microneedle adapter comprises a block of polymer material, and wherein the septum-penetrating hollow projection is formed from polymer material integrally with the block.

According to a further feature of an embodiment of the present invention, there is also provided a cap secured over the microneedle adapter, wherein the cap is configured to form a sterility-maintaining barrier around the at least one microneedle, and wherein the interengagement of the microneedle adapter with the syringe is configured to maintain sterility of the flow path from the septum-penetrating hollow projection to the at least one hollow microneedle.

According to a further feature of an embodiment of the present invention, there is also provided a sterility-maintaining wrapper enveloping the device with the liquid inside the syringe and with the microneedle adapter interengaged with the syringe in the storage position.

According to a further feature of an embodiment of the present invention, the syringe has a syringe body integrally formed from an impermeable polymer, the syringe body comprising: (a) a hollow barrel at least partially defining a liquid-receiving volume, the hollow barrel having a central axis; and (b) a threaded engagement configuration circumscribing the septum, wherein the septum is integrated with an axial region of the hollow barrel by overmolding.

There is also provided according to the teachings of an embodiment of the present invention, a device for delivering a liquid into the skin of a patient, the device comprising: (a) a syringe containing a liquid, the syringe having a syringe body integrally formed from an impermeable polymer, the syringe body comprising: (i) a hollow barrel at least partially defining a liquid-receiving volume, the hollow barrel having a central axis, (ii) a septum-penetrating hollow projection extending along the central axis and defining an outlet flow path from the liquid-receiving volume, and (iii) an engagement configuration circumscribing the outlet flow path; and (b) a microneedle adapter comprising: (i) a complementary engagement configuration configured for engaging the engagement configuration so as to define a path of motion of the microneedle adapter relative to the syringe body from a storage position to an actuated position, (ii) at least one hollow microneedle deployed for insertion into the skin of the patient, the at least one hollow microneedle being in fluid flow connection to a flow path passing through the microneedle adapter, and (iii) a septum deployed in facing relation to the septum-penetrating hollow projection, wherein, when the microneedle adapter is in the storage position, the fluid flow outlet is sealed by the septum and, when the microneedle adapter is displaced to the actuated position, the septum-penetrating hollow projection penetrates the septum, thereby opening a continuous fluid flow path for the liquid from the syringe through the microneedle adapter for injection through the at least one hollow microneedle.

According to a further feature of an embodiment of the present invention, the engagement configuration is implemented as a collar formed with an inwardly-facing threaded channel.

According to a further feature of an embodiment of the present invention, the syringe body is formed from a material selected from the group consisting of: a cyclic olefin copolymer and a cyclic olefin polymer.

According to a further feature of an embodiment of the present invention, there is also provided a sterility-maintaining wrapper enveloping the device with the liquid inside the syringe and with the microneedle adapter interengaged with the syringe in the storage position.

### BRIEF DESCRIPTION OF THE DRAWINGS

The invention is herein described, by way of example only, with reference to the accompanying drawings, wherein:

FIG. 1A is an isometric view of a prefilled syringe device with a microneedle interface, constructed and operative according to the teachings of an aspect of the present invention;

FIG. 1B is an enlarged isometric view of a microneedle adapter and cap from the device of FIG. 1A;

FIG. 1C is an enlarged isometric view of the microneedle adapter and cap of FIG. 1B separated to reveal the microneedle interface;

FIG. 1D is a magnified isometric view illustrating a preferred form of microneedles for use in the device of FIG. 1A;

FIG. 2 is a cross-sectional view taken along a central axis of the device of FIG. 1A;

FIGS. 3A and 3B are enlarged views of the region of FIG. 2 designated III, showing a microneedle adapter in a storage position and in an activated position, respectively;

FIG. 4 is an isometric view of a prefilled syringe device with a microneedle interface, constructed and operative according to the teachings of a further aspect of the present invention;

FIG. 5A is an enlarged isometric view of a microneedle adapter and cap from the device of FIG. 4;

FIG. 5B is an enlarged isometric view of a syringe body from the device of FIG. 4;

FIG. 6 is a cross-sectional view taken along a central axis of the device of FIG. 4; and

FIGS. 7A and 7B are enlarged views of the region of FIG. 6 designated VII, showing a microneedle adapter in a storage position and in an activated position, respectively.

### DESCRIPTION OF THE PREFERRED EMBODIMENTS

The present invention is a prefilled syringe device employing a microneedle interface for intradermal delivery.

The principles and operation of devices according to the present invention may be better understood with reference to the drawings and the accompanying description.

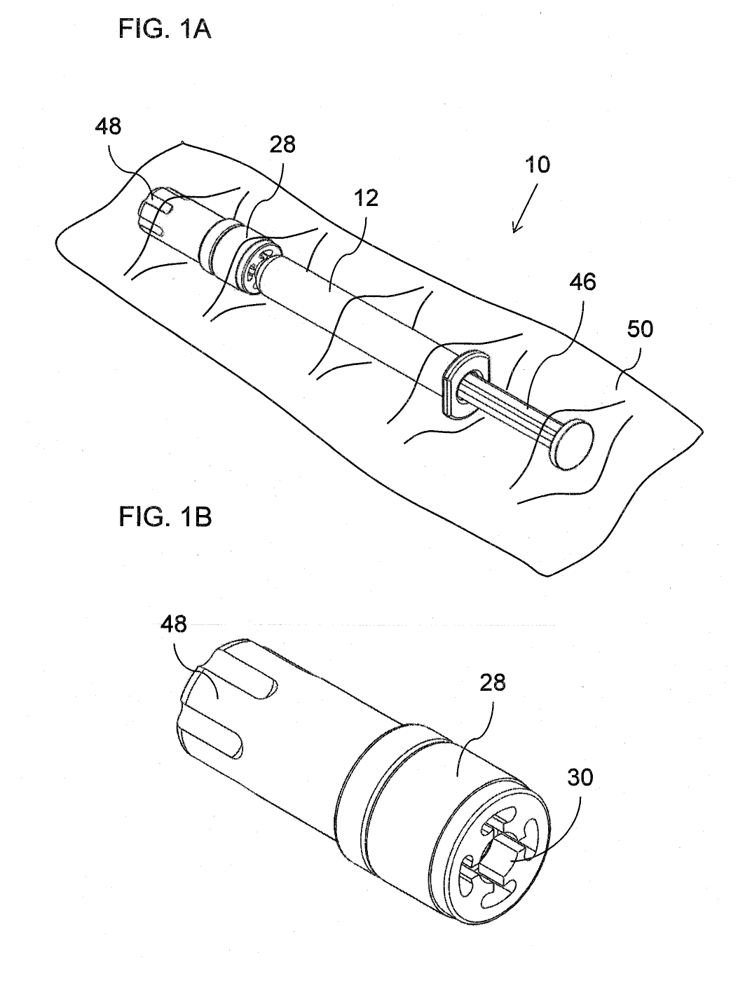

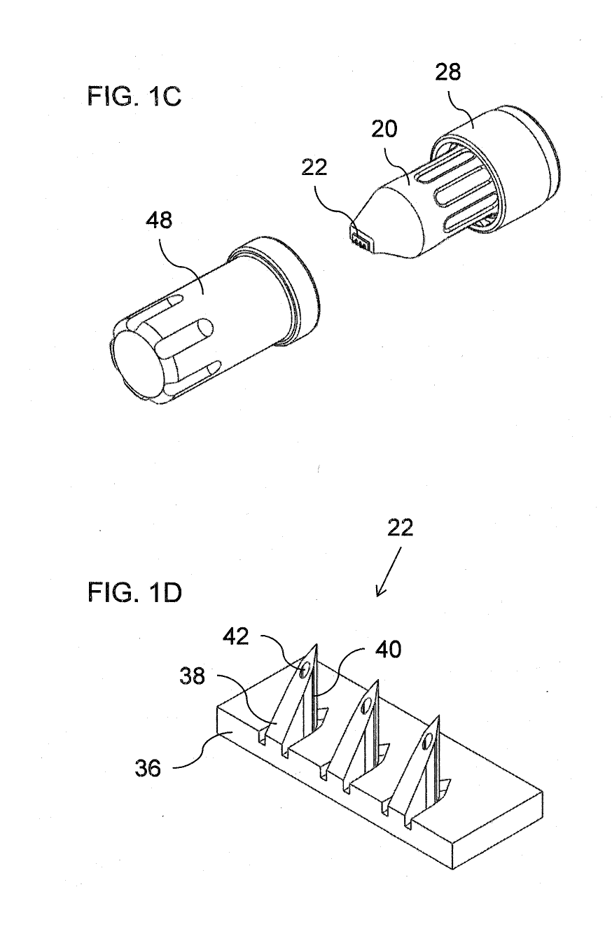

Referring now to the drawings, FIGS. 1A-3B illustrate a device, generally designated 10, constructed and operative according to the teachings of a first aspect of the present invention, for delivering a liquid into the skin of a patient. Generally speaking, device 10 includes a syringe 12 containing a liquid 14 (best seen in FIG. 2), with a fluid flow outlet 16. A septum 18 is attached to syringe 12 in sealing relation to fluid flow outlet 16.

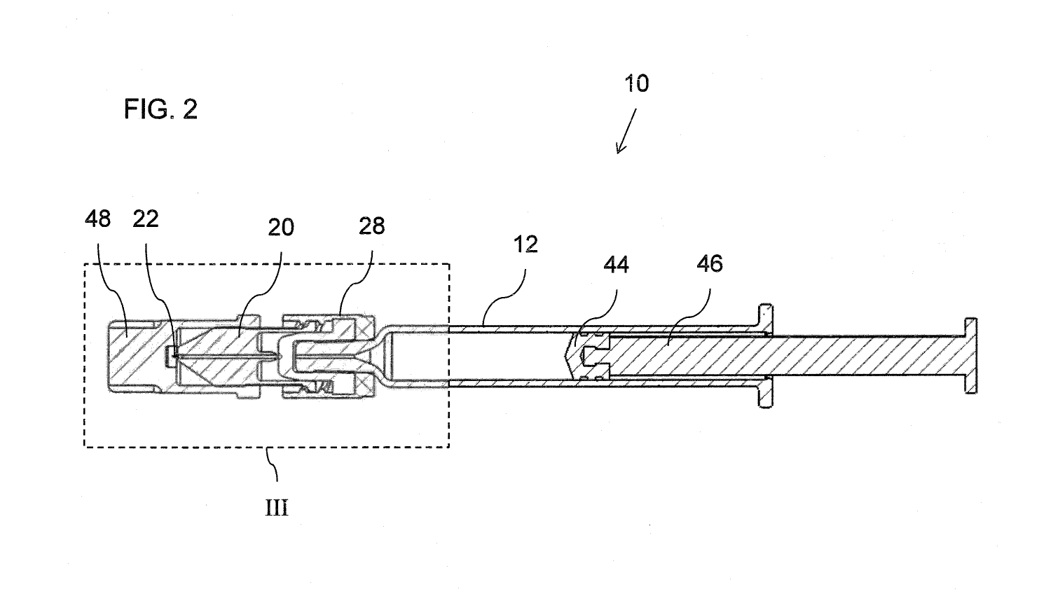

Device 10 also includes a microneedle adapter 20 interengaged with syringe 12 so as to be displaceable from a storage position (FIG. 3A) to an actuated position (FIG. 3B). Microneedle adapter 20 includes at least one hollow microneedle 22 deployed for insertion into the skin of the patient, and a septum-penetrating hollow projection 24 deployed in facing relation to septum 18. Microneedle adapter 20 has an internal channel 26 defining a flow path from septum-penetrating hollow projection 24 to hollow microneedle(s) 22.

As a result of this structure, when microneedle adapter 22 is in the storage position of FIG. 3A, fluid flow outlet 16 is sealed by septum 18, thereby maintaining sealed storage conditions for liquid 14. When microneedle adapter 20 is displaced to the actuated position of FIG. 3B, septum-penetrating hollow projection 24 penetrates septum 18, thereby opening a continuous fluid flow path for the liquid from syringe 12 through microneedle adapter 20 for injection through hollow microneedle(s) 22.

At this stage, it will be appreciated that device 10 is particularly advantageous, providing a prefilled syringe solution for microneedle intradermal delivery where the liquid to be delivered is stored without exposure to any metal needle. This and other advantages of this aspect of the present invention will be discussed further below.

In the example illustrated here, septum 18 is attached to syringe 12 by a connecting collar 28, which fastens securely to syringe 12 and also provides a configuration for interengaging with microneedle adapter 20. In this example, securing of collar 28 to syringe 12 is achieved using a resilient locking configuration 30, best seen in FIG. 1B, which locks behind a recessed step at the rear of a Luer connector or other complementary syringe outlet configuration, thereby securing connecting collar 28 in place. According to one preferred manufacturing implementation, septum 18 is integrated with the connecting collar by overmolding, i.e., a two-stage molding process in which silicone rubber is injection molded into the required location while connecting collar 28 is still within the mold where it was formed. Alternatively, any alternative form of mechanical retention of septum 18 may be used.

In this implementation, connecting collar 28 also provides an engagement configuration cooperating with complementary features of microneedle adapter 20 to retain microneedle adapter 20 connected to syringe 12 in the storage position, and to allow relative motion of microneedle adapter 20 relative to syringe 12 from the storage position to the activated position. Motion between the storage position and the activated position preferably occurs by bringing together the two components in the direction of a central axis of syringe 12. Examples of engagement configurations which allow suitable ranges of motion include, but are not limited to, threaded engagement and a bayonet engagement. In the particularly preferred implementation illustrated here, connecting collar 28 is formed with a cylindrical outer wall from which helical internal threading 32 projects inwards. The threading is shown here as a double helix thread into which engage corresponding tabs 34 of microneedle adapter 20. In this case, displacement of microneedle adapter 20 relative to syringe 12 from the storage position to the activated position is achieved conveniently and reliably by rotation of microneedle adapter 20 about the longitudinal central axis of syringe 12. Most preferably, a resilient tooth or the like (not shown) at the beginning of the thread is deployed to oppose inadvertent separation of microneedle adapter 20 from syringe 12 prior to use.

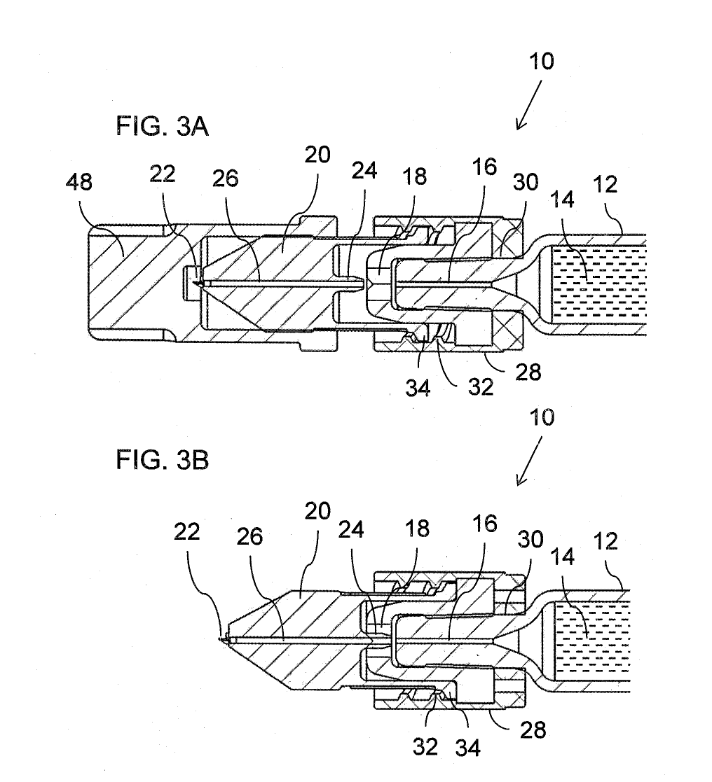

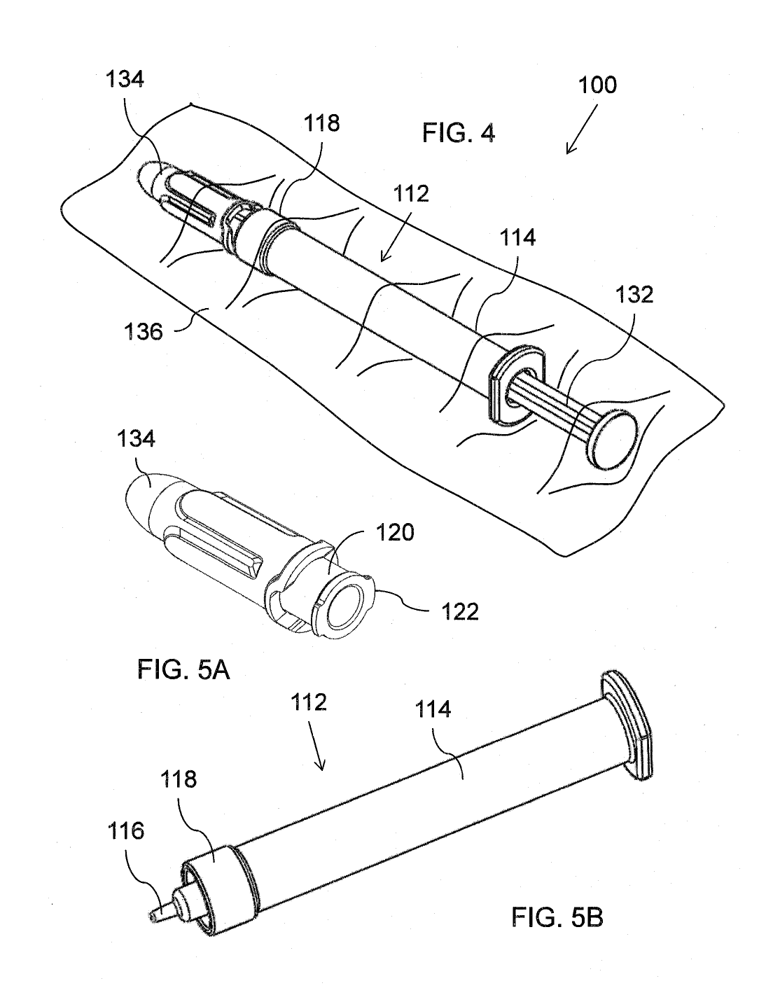

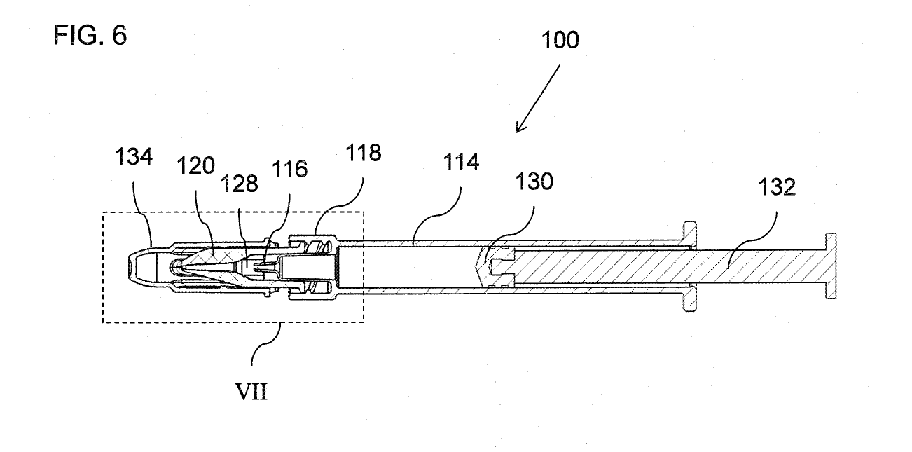

Parenthetically, it should be noted that, in an alternative preferred implementation, syringe 12 may be integrally formed from impermeable polymer material with a form structurally and functionally equivalent to connecting collar 28 by suitable injection molding production techniques, in a manner analogous to that described below with reference to the embodiment of FIGS. 4-7B. For example, in such an implementation, syringe 12 includes a syringe body, integrally formed from an impermeable polymer, and preferably including a hollow barrel and a threaded engagement configuration circumscribing the septum. Optionally, the septum may be integrated with an axial region of the hollow barrel by overmolding.

Turning now to microneedle adapter 20, in a particularly preferred implementation as illustrated, the body of the microneedle adapter is formed from a block of polymer material with which septum-penetrating hollow projection 24 is integrally formed. The use of an integrated polymer septum-penetrating element has advantages of simplicity of manufacture and assembly. Optionally, septum 18 may have a pre-formed partial or complete slit to facilitate penetration by projection 24.

The at least one microneedle 22 is most preferably implemented as at least two, and preferably at least three, microneedles 22 deployed in a linear array. Particularly preferred implementations employ MicroPyramid™ hollow microneedles commercially available from NanoPass Technologies (Israel), illustrated in FIG. 1D. These microneedle(s) are integrally formed with a substrate 36, and have an oblique surface 38 which intersects with a set of upright surfaces 40, and a fluid flow bore 42 intersecting with oblique surface 38.

It is a particularly preferred feature of certain embodiments of the present invention that the device is supplied as a prefilled syringe, pre-assembled and ready for use. Thus, referring primarily to FIGS. 1A and 2, device 10 is provided with the liquid to be delivered already loaded into the body of syringe 12 and retained by a piston 44 associated with a plunger 46. At the tip of the device, microneedle(s) 22 are preferably protected prior to use by a cap 48 secured over at least part of microneedle adapter 20. Cap 48 is preferably configured to form a sterility-maintaining barrier around microneedle(s) 22, and the interengagement of the microneedle adapter 20 with syringe 12 is preferably configured to maintain sterility of the flow path from septum-penetrating hollow projection 24 to microneedle(s) 22. Additionally, or alternatively, the prefilled and assembled device 10 is preferably enveloped prior to use in a sterility-maintaining wrapper 50 (FIG. 1A).

Most preferably, cap 48 is formed with sufficient clearance (see FIG. 2) to allow displacement of microneedle adapter 20 to its activated position by rotation of cap 48 prior to removal of the cap, thereby protecting microneedle(s) 22 until immediately prior to use.

Turning now to a further aspect of the present invention, FIGS. 4-7B illustrate a device 100 for delivering a liquid into the skin of a patient. Device 100 differs from device 10 primarily in that it employs a syringe body 112 integrally formed from an impermeable polymer so as to integrate a hollow barrel 114 with a septum-penetrating hollow projection 116 defining an outlet flow path and an engagement configuration 118 circumscribing the outlet flow path. The integration of these features into a single element, typically formed by injection molding techniques, provides a highly cost effective and reliable solution for implementing prefilled syringes with microneedle adapters.

This aspect of the present invention employs what are referred to herein as “impermeable polymers”. The term “impermeable” in this context refers to polymer materials which provide a sufficiently low level of permeability to liquid solutions that they are acceptable for use in prefilled syringe applications as specified in current industry standards, guidelines and Good Manufacturing Practices. As an alternative informal definition for the purpose of this document, “impermeable” in this context may be considered to refer to materials with the ability to store liquid solutions (for example, water-based solutions or emulsions) in a syringe for a period of at least 6 months, and preferably in excess of 1 year, without clinically relevant degradation of the solution. To be suitable for PFS applications, the material must also satisfy additional requirements regarding non-absorption of compounds from the solution and not releasing compounds into the solution, all as set out in the relevant ISO standards for “primary containers” for medical solutions. Various polymer materials are known in the art as suitable for such applications. Particularly preferred examples include, but are not limited to, a cyclic olefin copolymer (COC) and a cyclic olefin polymer (COP).

Production techniques for syringe body 112 are typically injection molding techniques, dictated by the desired structural features of the device as will be understood by one ordinarily skilled in the art of injection molding, and are per se standard techniques which will not be described here in detail.

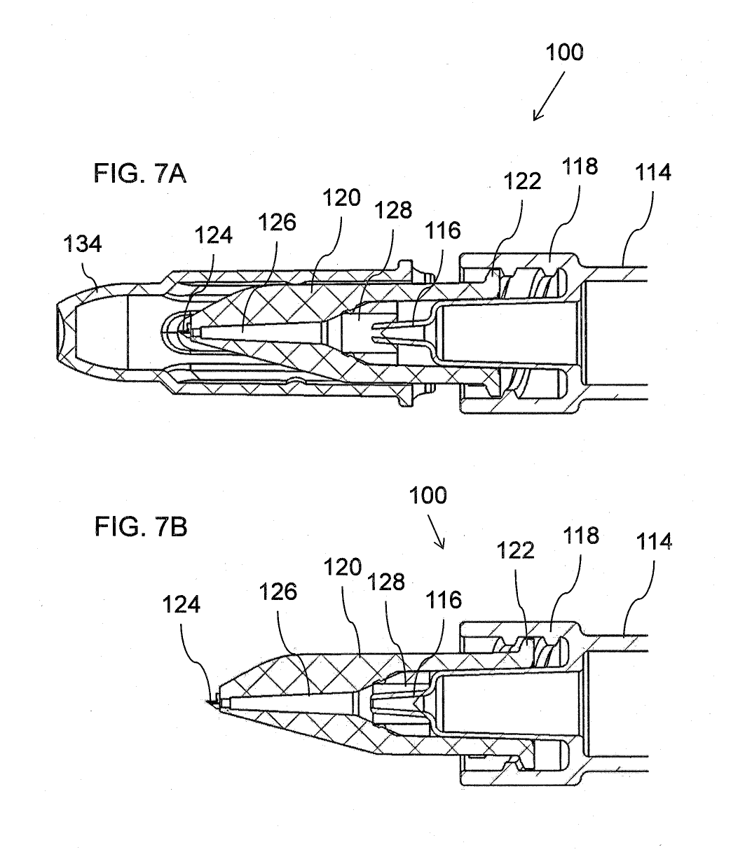

In cooperation with syringe body 112, device 100 also includes a microneedle adapter 120 which includes a complementary engagement configuration 122 configured for engaging engagement configuration 118 so as to define a path of motion of microneedle adapter 120 relative to syringe body 114 from a storage position (FIG. 7A) to an actuated position (FIG. 7B). Microneedle adapter 120 is provided with one or more hollow microneedles 124, in fluid flow connection to a flow path 126 passing through microneedle adapter 120. A septum 128 is deployed in facing relation to septum-penetrating hollow projection 116.

When microneedle adapter 120 is in the storage position of FIG. 7A, the fluid flow outlet of syringe 112 is sealed by septum 128, thereby defining (together with a piston 130 associated with a plunger 132) a primary container for long term storage of liquid. Prior to intradermal administration, microneedle adapter 120 is displaced relative to syringe body 112 to the actuated position (FIG. 7B), causing septum-penetrating hollow projection 116 to penetrate septum 128, thereby opening a continuous fluid flow path for the liquid from the liquid-receiving volume within syringe barrel 114 through microneedle adapter 120 for injection through hollow microneedles 124.

The remaining features of device 100 will be understood by analogy to the corresponding features of device 10 described above. For example, here too, a wide range of options exist for the implementation of engagement configuration 118 and complementary engagement configuration 122, including but not limited to, threaded engagement and bayonet connections. Here too, the device is illustrated in a particularly preferred configuration in which engagement configuration 118 is implemented as a cylindrical collar formed with an inwardly-facing double-helix threaded channel into which tabs of complementary engagement configuration 122 engage. One or more resilient tooth (not shown) or other suitable retaining features are deployed to resist removal of microneedle adapter 120 from syringe body 112.

Here too, a cap 134 is provided to protect microneedles 124 prior to use, and may also contribute to maintaining sterility of the adapter prior to use. Outer packaging 136 in the form of a sterility-maintaining wrapper envelopes the assembled device 100 complete with the contained liquid, with microneedle adapter 120 interengaged with syringe body 112 in the storage position, for long term storage until required for use.

It will be appreciated that the above descriptions are intended only to serve as examples, and that many other embodiments are possible within the scope of the present invention as defined in the appended claims.

What is claimed is:

1. A device for delivering a liquid into the skin of a patient, the device comprising:
(a) a syringe containing a liquid, said syringe including a fluid flow outlet;
(b) a septum attached to said syringe in sealing relation to said fluid flow outlet; and
(c) a microneedle adapter interengaged with said syringe so as to be displaceable from a storage position to an actuated position, said microneedle adapter comprising:
(i) at least one hollow microneedle deployed for insertion into the skin of the patient, and
(ii) a septum-penetrating hollow projection deployed in facing relation to said septum,
wherein said microneedle adapter defines a flow path from said septum-penetrating hollow projection to said at least one hollow microneedle,
wherein, when said microneedle adapter is in said storage position, said fluid flow outlet is sealed by said septum and, when said microneedle adapter is displaced to said actuated position, said septum-penetrating hollow projection penetrates said septum, thereby opening a continuous fluid flow path for said liquid from said syringe through said microneedle adapter for injection through said at least one hollow microneedle.

2. The device of claim 1, wherein said septum is attached to said syringe by a connecting collar, and wherein said microneedle adapter is interengaged with said syringe by engagement with said connecting collar.

3. The device of claim 2, wherein said septum is integrated with said connecting collar by overmolding.

4. The device of claim 1, wherein said microneedle adapter is interengaged with said syringe via a threaded engagement such that rotation of said microneedle adapter about a longitudinal axis of said syringe generates axial motion of said microneedle adapter from said storage position to said actuated position.

5. The device of claim 1, wherein said microneedle adapter comprises a block of polymer material, and wherein said septum-penetrating hollow projection is formed from polymer material integrally with said block.

6. The device of claim 1, further comprising a cap secured over said microneedle adapter, wherein said cap is configured to form a sterility-maintaining barrier around said at least one microneedle, and wherein the interengagement of said microneedle adapter with said syringe is configured to maintain sterility of said flow path from said septum-penetrating hollow projection to said at least one hollow microneedle.

7. The device of claim 1, further comprising a sterility-maintaining wrapper enveloping the device with said liquid inside said syringe and with said microneedle adapter interengaged with said syringe in said storage position.

8. The device of claim 1, wherein said syringe has a syringe body integrally formed from an impermeable polymer, said syringe body comprising:
(a) a hollow barrel at least partially defining a liquid-receiving volume, said hollow barrel having a central axis; and
(b) a threaded engagement configuration circumscribing said septum, wherein said septum is integrated with an axial region of said hollow barrel by overmolding.

9. A device for delivering a liquid into the skin of a patient, the device comprising:
(a) a syringe containing a liquid, said syringe having a syringe body integrally formed from an impermeable polymer, said syringe body comprising:
(i) a hollow barrel at least partially defining a liquid-receiving volume, said hollow barrel having a central axis,
(ii) a septum-penetrating hollow projection extending along said central axis and defining an outlet flow path from said liquid-receiving volume, and
(iii) an engagement configuration circumscribing said outlet flow path; and
(b) a microneedle adapter comprising:
(i) a complementary engagement configuration configured for engaging said engagement configuration so as to define a path of motion of said microneedle adapter relative to said syringe body from a storage position to an actuated position,
(ii) at least one hollow microneedle deployed for insertion into the skin of the patient, said at least one hollow microneedle being in fluid flow connection to a flow path passing through said microneedle adapter, and
(iii) a septum deployed in facing relation to said septum-penetrating hollow projection,
wherein, when said microneedle adapter is in said storage position, said fluid flow outlet is sealed by said septum and, when said microneedle adapter is displaced to said actuated position, said septum-penetrating hollow projection penetrates said septum, thereby opening a continuous fluid flow path for said liquid from said syringe through said microneedle adapter for injection through said at least one hollow microneedle.

10. The device of claim 9, wherein said engagement configuration is implemented as a collar formed with an inwardly-facing threaded channel.

11. The device of claim 9, wherein said syringe body is formed from a material selected from the group consisting of: a cyclic olefin copolymer and a cyclic olefin polymer.

12. The device of claim 9, further comprising a sterility-maintaining wrapper enveloping the device with said liquid inside said syringe and with said microneedle adapter interengaged with said syringe in said storage position.
